(19) United States
(12) Patent Application Publication
(10) Pub. No.: US 2008/0262616 A1
(43) Pub. Date: Oct. 23, 2008

(54) OSTEOCHONDRAL GRAFT AND METHOD OF USE FOR REPAIRING AN ARTICULAR CARTILAGE DEFECT SITE

(75) Inventor: William F. McKAY, Memphis, TN (US)

Correspondence Address: HESLIN ROTHENBERG FARLEY & MESITI P.C., 5 COLUMBIA CIRCLE, ALBANY, NY, 12203, US

(73) Assignee: WARSAW ORTHOPEDIC, INC., Warsaw, IN (US)

(21) Appl. No.: 11/736,914

(22) Filed: Apr. 18, 2007

(51) Int. Cl. A61F 2/08

(52) U.S. Cl. 623/14.12

(57) ABSTRACT

An osteochondral graft for use in repairing an articular cartilage defect site includes a cartilage cap and a wall extension. The cartilage cap has a top surface and a bottom surface with the wall extension projecting from the bottom surface. The wall extension includes an external surface and an internal surface with the internal surface defining an internal space. At least one of the internal surface or external surface may be tapered at an angle to facilitate implantation of the graft. The graft may be fabricated from collagen and configured as a composite construct, the cartilage cap being made from a porous collagen material to facilitate cartilage regeneration and the wall extension being made from a substantially cross-linked collagen to provide structural strength to facilitate insertion of the graft into the articular cartilage defect site. A method of repairing an articular cartilage defect with the osteochondral graft is also disclosed.

### CROSS-REFERENCE TO RELATED APPLICATION

This application contains subject matter which is related to the subject matter of the following application, which is hereby incorporated herein by reference in its entirety:

“Shaped Osteochondral Grafts and Methods of Using Same,” by McKay, U.S. Ser. No. 11/120,136, filed Apr. 30, 2005.

### TECHNICAL FIELD

The present invention relates generally to the field of grafting for articular cartilage repair, and more particularly, to a novel configured osteochondral graft and its use in articular cartilage resurfacing repair of a host defect site in a mammal.

### BACKGROUND OF THE INVENTION

Human joint surfaces are covered by articular cartilage that provides a resilient, durable surface with low friction. Cartilage is an avascular tissue that has a small number of chondrocytes encapsulated within an extensive extracellular matrix. The cartilage acts to distribute mechanical forces and to protect subchondral bone. For example, the knee is a particular instance of a cartilage surfaced (the condyle) bone area. The knee comprises three bones—the femur, tibia, and patella that are held in place by various ligaments. Corresponding chondral areas of the femur and the tibia form a hinge joint and the patella acts to protect the joint. Portions of the chondral areas as well as the undersurface of the patella are covered with articular cartilage that allows the femur, patella and tibia to smoothly glide against each other without causing damage.

Damage to the articular cartilage, subchondral bone or both can result from traumatic injury or a disease state. For example, articular cartilage in the knee can be damaged due to traumatic injury as with athletes and via a degenerative process as with older patients. The knee cartilage does not heal well due to the lack of vascularity. Hyaline cartilage in particular has a limited capacity for repair and lesions in this material, without intervention, can form scar tissue lacking the biomechanical properties of normal cartilage.

A number of procedures are used to treat damaged articular cartilage. Currently, the most widely used procedure involves lavage, arthroscopic debridement and repair stimulation. Repair stimulation is conducted by several methods including, drilling, abrasion arthroplasty and microfracture. The goal of these procedures is to penetrate into subchondral bone to induce bleeding and fibrin clot formation. This reaction promotes initial repair. However, the resulting formed tissue is often fibrous in nature and lacks the durability of normal articular cartilage.

In an attempt to overcome the problems associated with the above techniques, osteochondral transplantation, also known as “mosaicplasty” or “OATS” has been used to repair articular cartilage. This procedure involves removing injured tissue from the articular defect and drilling cylindrical openings in the area of the defect and underlying bone. Solid cylindrical plugs, consisting of healthy cartilage overlying subchondral bone, are harvested from another area of the patient, typically from a lower weight-bearing region of the joint under repair, or from a donor patient, and are implanted in the host openings. However, in these cases, if the opening is too large, the graft can rotate or move within the host site and become loose, which will prevent bio-ingrowth with the surrounding tissues. Further, if the host site is too small, significant tissue and cellular damage can occur to the graft during the implantation.

Historically, osteochondral grafting has been used successfully to repair chondral damage and to replace damaged articular cartilage and subchondral bone. First, in this procedure, cartilage and bone tissue of a defect site are removed by routing to create a cylindrical bore of a precise geometry. Then a cartilage and subchondral bone plug graft is harvested in a matching geometry. The donor plug graft is typically removed from another body region of less strain. The donor plug graft can be harvested from a recipient source (autograft) or from another suitable human or other animal donor (allograft and xenograft respectively). The harvested plug graft is then implanted into the bore of the routed defect site. Healing of the plug graft to the host bone results in fixation of the plug graft to the surrounding host region.

Success of the grafting process is dependant on the intimate seating and sizing of the graft within the socket. First, surface characteristics of the plug graft are critical. For the procedure to be successful, the surface of the transplanted plug graft must have the same contour as the excised osteochondral tissue. If the contour is not a correct match, a repaired articular surface is at risk for further damage during patient ambulation. Additionally, some graft shapes do not pack well into irregular defects. The graft may have a propensity to rotate resulting in poor integration of the graft to the surrounding host tissue. An improperly placed and sized plug graft can result in host tissue integration failure and post implantation motion with associated articular surface collapse.

Accordingly, there is a need for an improved and/or alternative shaped osteochondral graft and an associated implantation technique, for repairing articular cartilage defects.

### SUMMARY OF THE INVENTION

The present invention comprises an osteochondral graft for use in repairing an articular cartilage defect site in a mammal. The osteochondral graft disclosed herein employs a cartilage cap that has a certain thickness with top and bottom surfaces and a wall extension projecting from the cartilage cap. The wall extension has an external surface and an internal surface, with the internal surface at least partially defining an internal space. The osteochondral graft is implanted to repair an articular cartilage defect site following the removal of damaged and/or diseased articular cartilage.

More particularly, the present invention provides in one aspect, an osteochondral graft configured for repairing an articular cartilage defect site. The osteochondral graft includes a cartilage cap and a wall extension, with the cartilage cap having a top surface and a bottom surface and the wall extension projecting from the bottom surface of the cartilage cap. The wall extension includes an external surface and an internal surface with the internal surface at least partially defining a centralized internal space. The wall extension and internal space are sized and configured to facilitate the implantation of the osteochondral graft into the articular cartilage defect site.

The present invention provides in another aspect, an osteochondral graft configured for repairing an articular cartilage defect site. The osteochondral graft includes a cartilage cap and a wall extension, with the cartilage cap having a top surface and a bottom surface, with the wall extension projecting from the bottom surface of the cartilage cap. The wall extension includes an external surface and an internal surface with the internal surface at least partially defining a centralized internal space. The osteochondral graft further includes a central axis that extends between the cartilage cap and the proximal end of the wall extension. Either the internal surface and/or the external surface may be tapered at a taper angle relative to the central axis. This taper angle assists the user when inserting the osteochondral graft into the surgically prepared articular cartilage defect site.

Another aspect of the present invention provides a method for repairing an articular cartilage defect site in a mammal, the method includes surgically creating an opening in the articular cartilage defect site. The opening includes a centrally positioned platform and a channel that is circumferential relative to the platform and extends to a certain depth below the articular cartilage defect site. The method also includes employing an osteochondral graft that includes a cartilage cap and a wall extension. The cartilage cap has a top surface and a bottom surface with the wall extension projecting from the bottom surface of the cartilage cap. The wall extension also has an external surface and an internal surface. The internal surface at least partially defines or bounds a centralized internal space. The method provides further for implanting the osteochondral graft into the opening. The osteochondral graft is properly sized and configured to be inserted into the surgically created opening. After being implanted, the bottom surface of the cartilage cap of the osteochondral graft will contact the centralized platform of the opening and the wall extension will have slid into the channel of the opening. The configuration of the cartilage cap and the wall extension will promote and enhance bio-ingrowth between the implanted osteochondral graft and the opening.

Further, additional features and advantages are realized through the techniques of the present invention. Other embodiments and aspects of the invention are described in detail herein and are considered a part of the claimed invention.

### BRIEF DESCRIPTION OF THE DRAWINGS

The subject matter which is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1 is a perspective view of one embodiment of an osteochondral graft, shown before being implanted into prepared host site, in accordance with an aspect of the present invention;

FIG. 2 is a cross-sectional, side elevational view of the osteochondral graft and the corresponding prepared articular cartilage defect site of FIG. 1 taken along line 2-2, in accordance with an aspect of the present invention;

FIG. 3 is a perspective view of the osteochondral graft and the correspondingly dimensioned articular cartilage defect site of FIG. 1 shown with a tab and an aligned corresponding slot within the surgically prepared opening of the defect site, before implantation of the osteochondral graft, in accordance with an aspect of the present invention;

FIG. 4 is a cross-sectional, side elevational view of one embodiment of an osteochondral graft, showing an inside surface of a wall extension with a tapered angle and the corresponding prepared articular cartilage defect site, in accordance with an aspect of the present invention;

FIG. 5 is a cross-sectional, side elevational view of one embodiment of an osteochondral graft, showing an outside surface of a wall extension with a tapered angle and the corresponding prepared articular cartilage defect site, in accordance with an aspect of the present invention;

FIG. 6 is a cross-sectional, side elevational view of the osteochondral graft of FIG. 1 showing the bilayered collagen construct, in accordance with an aspect of the present invention;

FIG. 7A is a perspective view of the osteochondral graft of FIG. 1, shown with at least one hole extending from the inside surface to the outside surface of the wall extension, in accordance with an aspect of the present invention;

FIG. 7B is a perspective view of the osteochondral graft of FIG. 1, shown with at least one slot extending in a distal direction from an end of the wall extension and through from the inside surface to the outside surface, in accordance with an aspect of the present invention;

FIG. 8A is a perspective view of a coring tool prior to insertion into an opening in the articular cartilage defect site to create a central platform and a peripheral channel for accommodating the inserted osteochondral graft, in accordance with an aspect of the present invention;

FIG. 8B is a cross-sectional, side elevational view of the coring tool of FIG. 8A shown with the planar cutting surface and extending peripheral cutting teeth, in accordance with an aspect of the present invention;

FIG. 8C is a perspective view of a punch tool inserted into the opening in the articular cartilage defect site for creating at least one slot in a side wall of the opening, in accordance with an aspect of the present invention;

FIG. 8D is a perspective view of the osteochondral graft partially inserted into the surgically created opening, in accordance with an aspect of the present invention; and

FIG. 9 is a perspective of one embodiment of two interlocked osteochondral grafts, shown being implanted into prepared articular cartilage defect site, in accordance with an aspect of the present invention.

### BEST MODE FOR CARRYING OUT THE INVENTION

Generally stated, disclosed herein is an osteochondral graft that includes as components, a cartilage cap and a wall extension. Further described herein is a method of using an osteochondral graft for repairing (for example) an articular cartilage defect site located in the distal aspect of a femoral condyle.

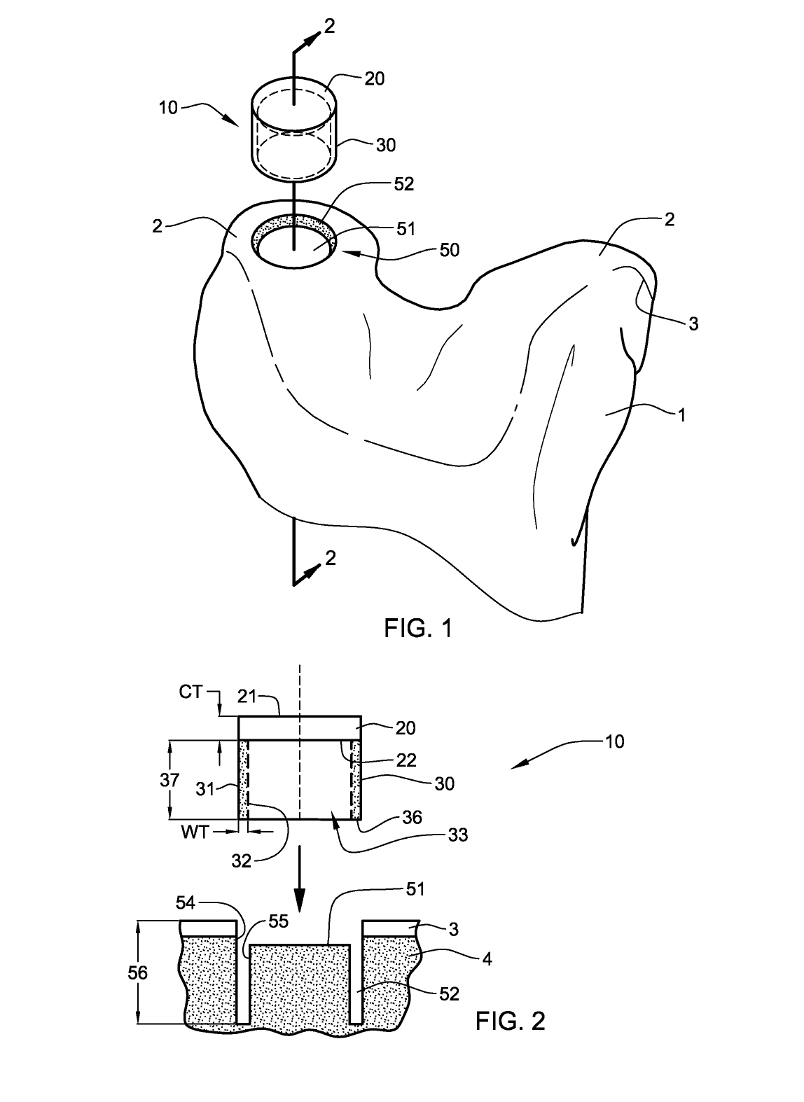

One embodiment of an osteochondral graft 10, in accordance with an aspect of the present invention, is illustrated in FIGS. 1-3 and described below.

As shown in FIG. 2, osteochondral graft 10 includes a cartilage cap 20 to which is attached a wall extension 30. Cartilage cap 20 has a top surface 21 and a bottom surface 22, with top surface 21 being generally comprised of articular (hyaline) cartilage 3. Bottom surface 22 composition will depend upon the cap thickness CT. Generally, bottom surface 22 will be comprised of subchondral bone 4, although when cap thickness CT is relatively thin, bottom surface 22 may include a matrix of articular cartilage 3 and subchondral bone 4. Cap thickness CT may range in valve from 1 to 10 millimeters with a more detailed range being 2 to 5 millimeters. Generally, cap thickness CT will be greater than the thickness of articular cartilage 3 to allow the user to remove a small amount of subchondral bone 4 to cause bleeding and a release of cells to stimulate bony repair. Wall extension 30 projects generally from the outer peripheral portion of bottom surface 22 of cartilage cap 20 and includes an external surface 31 and an internal surface 32. External surface 31 and internal surface 32 extend substantially parallel relative to each other and forming a wall extension thickness WT between them ranging from 1 to 10 millimeters with a more detailed range being 1 to 5 millimeters. An internal space 33 is defined by internal surface 32 and has a depth 37 that is measured from the proximal end 36 of wall extension 30. Wall extension 30 may completely circumvent internal space 33 or, alternatively only partially enclose (not shown) internal space 33. Depth 37 will generally vary and be determined by the user. Several factors may be evaluated by the user when determining depth 37, these include, but are not limited to, the quality of subchondral bone 4 of the defect site into which osteochondral graft 10 will be implanted, the structural dimensions of wall extension 30, and the structural integrity of the biomaterial that comprises wall extension 30.

FIG. 1 illustrates an example of one embodiment of osteochondral graft 10 of the present invention. The outer profile of graft or plug 10 is shaped like a cylindrical straight rod with a circular cross-sectional profile. The terms “plug” and “graft” may be used interchangeably herein to mean generally the osteochondral implant used to repair an articular cartilage. As shown, FIG. 1 is a perspective view of osteochondral graft 10 being aligned with a resultant opening 50, surgically created by the user following the removal of damaged articular cartilage from the surface of the condyle 2 of the femur 1. For the example seen in FIG. 1, the corresponding profile of opening 50 is circular with the opening perimeter being dimensioned to allow for implantation of the cylindrical rod shaped osteochondral graft 10. Receipt of the osteochondral graft 10 within corresponding opening 50 can also include an interference fit, if desired.

FIG. 2 provides a cross-sectional view of opening 50 following the surgical removal of damaged and/or diseased articular cartilage 3 and employment of a cutting tool 60 (not shown). For the example shown, opening 50 is configured to include a central platform 51 and a circumferential channel 52, circumferential channel 52 being oriented concentric to central platform 51. Although not shown, it should be noted that channel 52 may only partially circumvent platform 51 in the event osteochondral graft 10 is constructed of a correspondingly dimensioned (i.e. at least partial) wall extension 30. The width of channel 52 is dimensioned to accommodate wall thickness WT of wall extension 30, although it is contemplated that channel 52 may be sized to include an interference fit, if desired. Further, for the example shown in FIG. 2, the internal walls 54 of channel 52 and the sides 55 of platform 51 are substantially parallel relative to each other. As seen in FIG. 2, the cross-section profile and height of platform 51 are dimensioned to be received into internal space 33. Following the implantation of osteochondral graft 10 within opening 50, bottom surface 22 will generally contact the top portion of platform 51 or, alternatively will be in close approximation to allow for bio-ingrowth to occur between these two structures. Following implantation of osteochondral graft 10 into opening 50, platform 51 will function to provide support to cartilage cap 20 and, thereby eliminate several common post-operative complications including graft collapse, subchondral cyst formation and instability. Following final seating of osteochondral graft 10 within opening 50, top surface 21 will generally be positioned tangent to or flush with the surrounding host articular cartilage.

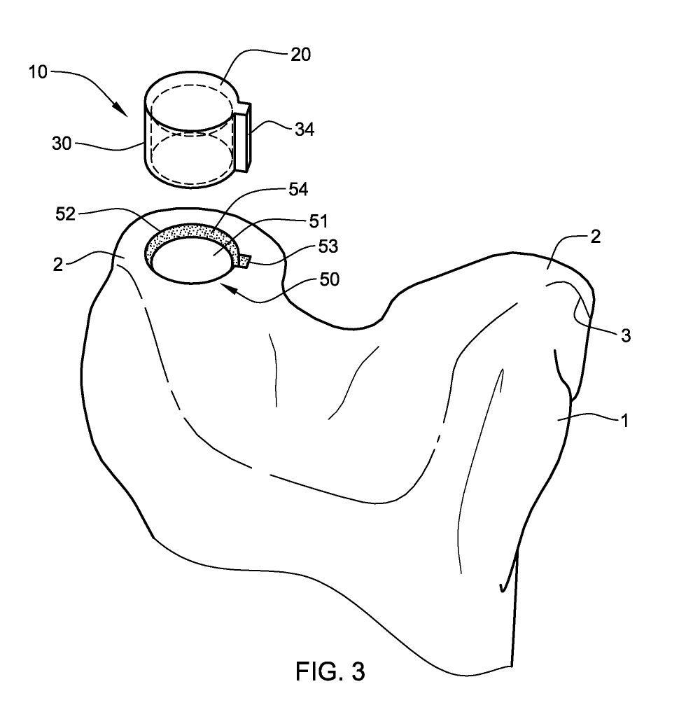

As shown in FIG. 3, opening 50 may also include a slot 53 or other shaped cut-out disposed along at least one wall 54 of channel 52. Slot 53 may either extend the entire depth 56 of channel 52 (not shown) or, alternatively only a portion of depth 56 (see FIG. 2). Slot 53 is configured to receive and mechanically restrain a corresponding tab 34 that projects away or radially from external surface 31 of wall extension 30. Tab 34 is disposed on external surface 31 and may either extend external surface's 31 full length or along only a part of external surface's 31 length. It should be understood to those skilled in the art that the configuration of tab 34 and corresponding slot 53 may be configured as a dovetail, longitudinal rod or other similar male-female undercut arrangements. Tab 34 and slot 53 function to mechanically secure osteochondral graft 10 within opening 50 to resist rotational and other loading forces that may occur during a patient's post-implantation ambulation. It also should be understood to those skilled in the art that it is contemplated that a plurality of such tab-slot/male-female interlocking mechanisms may be disposed along osteochondral plug 10 and channel wall 54 and that the number of such mechanisms used will generally be determined by the user. It should be further understood to those skilled in the art that the male-female interlocking mechanism may be reversed with the male member being positioned along channel wall 54 and the female member being disposed along external surface 31.

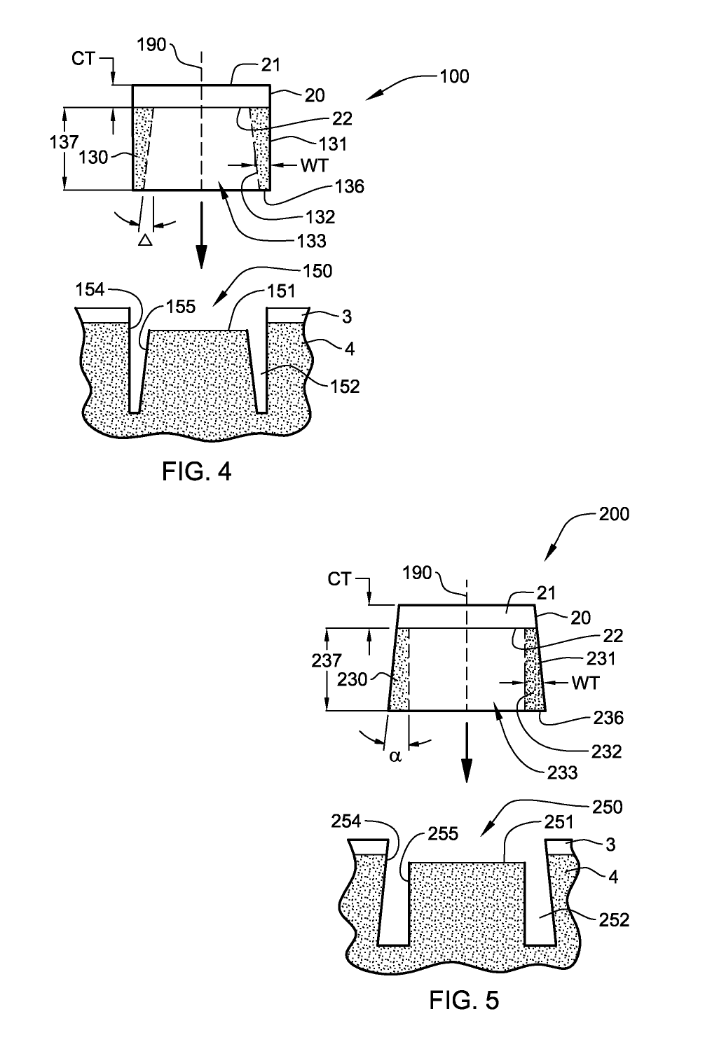

FIG. 4 shows a further alternative embodiment of the invention. As seen in the cross-sectional view of FIG. 4, osteochondral graft 100 includes cartilage cap 20 to which is attached a wall extension 130. As has been described previously above, cartilage cap 20 has top surface 21 and bottom surface 22, with top surface 21 being generally comprised of articular (hyaline) cartilage 3. Bottom surface 22 composition will depend upon the cap thickness CT for osteochondral graft 100 with bottom surface 22 being generally comprised of subchondral bone 4, although when cap thickness CT is relatively thin, bottom surface 22 may be include a matrix of articular cartilage 3 and subchondral bone 4. As stated above, generally cap thickness CT is greater than articular cartilage 3 to allow the user with the option of pre-implantation resection to produce a bleeding bone bed. Wall extension 130 projects generally from the peripheral portion of bottom surface 22 of cartilage cap 20 and includes an external surface 131 and an internal surface 132. External surface 131 extends in a substantially perpendicular direction from bottom surface 22. Internal surface 132 extends at a tapered angle A relative to a central axis 190. Taper angle A may range in value from 2 to 10 degrees with a more detailed range being 2 to 5 degrees. An internal space 133 is defined by internal surface 132 and has a depth 137 that is measured from the proximal end 136 of wall extension 130 to bottom surface 22. As discussed previously, although not shown, it is also contemplated that wall extension 130 and thus internal surface 137, may only partially circumvent internal space 133. As a result of the tapered orientation of internal surface 132, the area of internal space 133 near bottom surface 22 will be less than the area of internal space 133 near proximal end 136. Further, wall thickness WT will be greater distally than proximally. Again, depth 137 will generally vary and be determined by the user. As discussed above, several factors will be evaluated by the user when determining a final depth 137, these may include, but are not limited to, the quality of subchondral bone 4 of the defect site into which osteochondral graft 100 will be implanted, the structural dimensions of wall extension 130, and the structural integrity of the biomaterial that comprises wall extension 130.

FIG. 4 also provides a cross-sectional view of the corresponding profile of opening 150 following the surgical removal of the damaged and/or diseased articular cartilage 3 and the use of a surgical tool (not shown) for preparing the configuration of opening 150. Opening 150 is generally configured to include a central platform 151 and a circumferential channel 152, channel 152 being oriented concentric to central platform 151. Although not shown, it should be understood to those skilled in the art that channel 152 may only partially circumvent platform 151 in the event osteochondral graft 100 is constructed with a correspondingly dimensioned (i.e. partial) wall extension 130. The depth and width of channel 152 is dimensioned to accommodate wall extension 130, although it is contemplated that channel 152 may be sized to include an interference fit, if desired. Additionally, the platform sides 155 are tapered at an angle to accommodate the corresponding tapered orientation of internal surface 132. The channel walls 154 are oriented substantially parallel to central axis 190. Because of the tapered orientation of platform sides 155, the cross-sectional profile of platform 151 will be larger proximally then compared to the distal profile and such profile will be accommodated by internal space 133. The tapered configuration will assist in maintaining the structural strength of platform 151 prior to the implantation of osteochondral graft 100.

In addition, FIG. 4 illustrates platform 151 being dimensioned to be inserted into internal space 133 and following such insertion of osteochondral graft 100, for bottom surface 22 to generally contact the top portion of platform 151 or, alternatively to be in close approximation to allow for bio-ingrowth to occur between platform 151 and bottom surface 22. As described previously, following implantation of osteochondral graft 100 into opening 150, platform 151 will function to provide structural support to cartilage cap 20 and will contribute to reducing the occurrence of the several possible clinical complications described above. Following final seating of osteochondral graft 100 within opening 150, top surface 21 will generally be positioned flush with the surrounding remaining host articular cartilage.

Although not shown, it should be understood to those skilled in the art that osteochondral graft 100 and opening 150 may also be constructed with a similar male-female interlocking mechanism that has been described for osteochondral graft 10 previously herein, for maintaining among other things, rotational stability between osteochondral graft 100 and opening 150 following implantation.

FIG. 5 shows yet a further alternative embodiment of the invention. As seen in the cross-sectional view of FIG. 5, osteochondral graft 200 includes cartilage cap 20 to which is attached a wall extension 230. As has been described previously above, cartilage cap 20 has top surface 21 and bottom surface 22, with top surface 21 being generally comprised of articular (hyaline) cartilage 3. Bottom surface 22 composition will depend upon the cap thickness CT for osteochondral graft 200 with bottom surface 22 being generally comprised of subchondral bone 4, although when cap thickness CT is relatively thin, bottom surface 22 may be include a matrix of articular cartilages and subchondral bone 4. As provided above herein, cap thickness CT is generally thicker than articular cartilage layer 3. This dimensional difference provides the user with the ability to resection a portion of subchondral bone 4 from osteochondral graft 200 to obtain a bleeding bone bed to facilitate the repair process. Wall extension 230 projects generally from the peripheral portion of bottom surface 22 of cartilage cap 20 and includes an external surface 231 and an internal surface 232. External surface 231 extends at a tapered angle Δ relative to a central axis 190. Taper angle Δ may range in value from 2 to 10 degrees with a more detailed range being 2 to 5 degrees. Internal surface 232 extends in a substantially perpendicular direction from bottom surface 22 and is oriented substantially parallel to central axis 190. Wall thickness WT will be greater proximally when compared to distal wall thickness WT as a result of taper angle Δ. An internal space 233 is defined by internal surface 232 and has a depth 237 that is measured from the proximal end 236 of wall extension 230 to bottom surface 22. As discussed previously, although it is not shown, it is contemplated that wall extension 230 and thus internal surface 232, may only partially circumvent internal space 233. Again, depth 237 will generally vary and be determined by the user. As discussed previously, several factors will be evaluated by the user when determining a final depth 237, these may include, but are not limited to, the quality of subchondral bone 4 of the defect site into which osteochondral graft 200 will be implanted, structural dimensions of wall extension 230, and the structural integrity of the biomaterial that comprises wall extension 230.

FIG. 5 also provides a cross-sectional view of the corresponding profile of opening 250 following the surgical removal of the damaged and/or diseased articular cartilage 3 and the use of a surgical tool (not shown) to prepare the configuration of opening 250. Opening 250 is generally configured to include a central platform 251 and a circumferential channel 252, channel 252 being oriented concentric to central platform 251. Although not shown, it should be understood to those skilled in the art that channel 252 may only partially circumvent platform 251 in the event osteochondral graft 200 is constructed with a correspondingly dimensioned (i.e. partial) wall extension 230. The depth and width of channel 252 is dimensioned to accommodate the changing wall thickness WT of wall extension 230. It is also contemplated that channel 252 may be sized to include an interference fit, if desired. Additionally, the channel walls 254 are tapered at an angle to accommodate the tapered orientation of external surface 231. This tapered configuration may result in a gap between the external surface 231 and channel walls 254 following implantation of osteochondral graft 200 for the full or partial length of external surface 231. Because of the tapered orientation of channel walls 254, the cross-sectional profile of opening 250 will be larger proximally then compared to the distal profile. The platform sides 255 are oriented substantially parallel to central axis 190.

As seen in FIG. 5, the profile of platform 251 is configured to be inserted into internal space 233. Following the implantation of osteochondral graft 200, bottom surface 22 will contact the top part of platform 251 or, alternatively will be in close enough approximation to achieve bio-ingrowth between platform 251 and bottom surface 22. As described previously, following implantation of osteochondral graft 200 into opening 250, platform 251 will function to provide structural support to cartilage cap 20 and will assist in reducing the previously listed possible clinical complications. After final placement of osteochondral graft 200 within opening 250, top surface 21 will generally be positioned flush or tangent to the surrounding host articular cartilage.

Although not shown, it should be understood to those skilled in the art that osteochondral graft 200 and opening 250 may also be fabricated with a similar male-female interlocking mechanism that has been described for osteochondral graft 10 previously herein for resisting in vivo forces and maintaining rotational stability between osteochondral graft 200 and opening 250 after implantation.

FIG. 7A shows a perspective view of osteochondral graft 10 further including at least one proximal hole 35 extending from external surface 31 to internal surface 32. The size and position of proximal hole 35 will vary depending upon the quality of the subchondral bone of the host defect site into which osteochondral graft 10 is implanted.

As seen in FIG. 7B, it is also contemplated that osteochondral graft 10 may also include another opening in the proximal aspect of wall extension 30. A transverse slot 30 is shown that extends completely through wall extension 30 with the opening gap of slot 39 being disposed on end 35 of wall extension 30.

Both proximal hole 35 and slot 39 function to enhance fixation of osteochondral graft 10 within the host defect site by allowing bio-ingrowth to occur through the bore 38 of proximal hole 35 and the channel 43 of slot 39. Alternatively, bore 38 or channel 43 may be coated or treated with one of the later described bio-ingrowth facilitators. Although not shown, it should be understood to those skilled in the art that alternative osteochondral graft embodiments 100, 200 may also include in their respective constructs at least one proximal hole 35 and/or slot 39.

In certain aspects of the present invention, osteochondral graft 10, 100, 200 described herein for repairing an articular cartilage defect site may have a cross-sectional profile other than a circular cylinder as previously discussed. In some of the inventive embodiments described herein, such cross-sectional profiles will be that of a polygon, including equilateral and non-equilateral polygons, and regular and non-regular polygons. The polygon typically having from three to about ten sides, including e.g. triangles, rectangles, pentagons, hexagons, cruciforms, etc. In other embodiments, such cross-sectional profile will be non-circular, but will include at least one arc of a circle (sometimes herein referred to as a “circular arc”). Osteochondral grafts 10, 100, 200 having such shapes can be configured for receipt within surgically prepared openings in a human or other mammalian knee, hip or shoulder joints and will be capable of withstanding the biomechanical loads typically experienced within such joints without significant occurrence of fracture of the wall extension or collapse of the cartilage cap of the osteochondral graft.

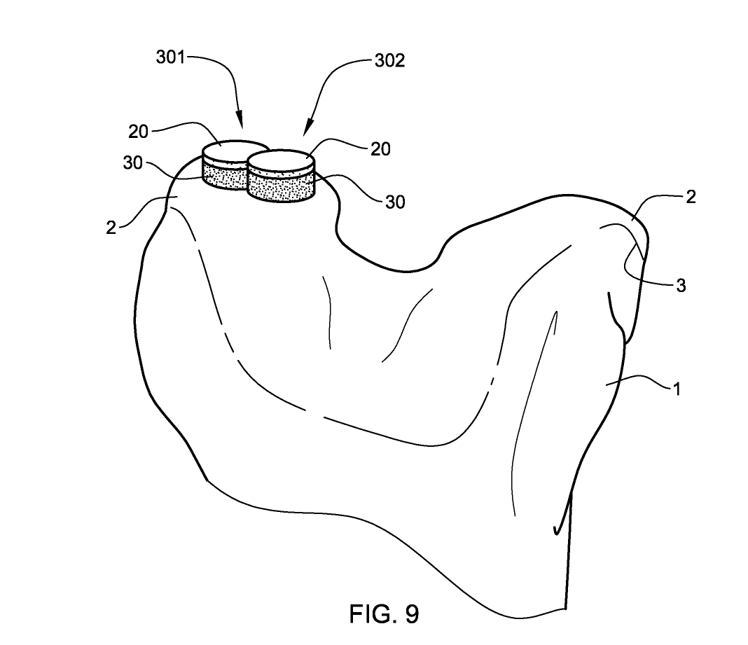

As described above, in certain aspects of the present invention, the osteochondral graft may be configured with cross-sectional profiles that are different than a circular cylinder to allow for mechanical interlocking with adjacent surfaces of the host defect site or, alternatively, other osteochondral grafts. An example of such an alternative embodiment is seen at FIG. 9. As shown, two osteochondral grafts 301, 302 have of cross sectional profiles that are partial circles with first osteochondral graft 301 including at least one circular arc that intersects with a circular arc of an adjoining or adjacent second osteochondral graft 302. It is contemplated that such mechanical interlocking arrangements may also include multiple osteochondral grafts that have cross-sectional profiles defined by multiple, intersecting circular arcs, e.g. two, three, four or more intersecting circular arcs. Further, although not shown, adjacent osteochondral grafts may have cross sectional polygonal profiles, including equilateral and non-equilateral polygons, and regular and non-regular polygons, including e.g. triangles, rectangles, pentagons, hexagons and cruciforms. The cross-sectional profile protrusions of such polygonal osteochondral grafts can be configured for receipt within surgically prepared openings in a human or other mammalian knee, hip or shoulder joints to provide a mechanically interlocked arrangement that is capable of withstanding the biomechanical loads typically experienced within such joints without significant occurrence of clinical complications.

The osteochondral grafts or plugs, a term often used by an artisan skilled in the art to describe a graft, of and for use in the various embodiments of the invention described herein can be harvested from the recipient as an autograft, from a suitable human as an allograft or from an animal donor as a xenograft. Either type of graft will need to be obtained from an appropriate structure including hyaline cartilage and underlying subchondral bone. Such suitable harvest locations in large part occur in weight bearing joints of mammals, including humans. These harvest locations include, for example, articular cartilage and rib cartilage. A wide variety of articular cartilages may be used including for example those taken from articulating surfaces of the knee, hip, or shoulder joints. As specific examples, osteochondral grafts or plugs may be taken from the femoral condyle, the articulating surfaces of the knee, or the articulating surfaces of the shoulder.

In the case of an allograft osteochondral graft or plug, these can be either fresh (containing live cells) or processed and frozen or otherwise preserved to remove cells and other potentially antigenic substances while leaving behind a scaffold for patient tissue bio-ingrowth. A variety of such processing techniques are known and can be used in accordance with the invention. For example, harvested osteochondral grafts or plugs can be soaked in an agent that facilitates removal of cell and proteoglycan components. One such solution that is known includes an aqueous preparation of hyaluronidase (type IV-s, 3 mg/ml), and trypsin (0.25% in monodibasic buffer 3 ml). The harvested osteochondral graft or plug can be soaked in this solution for several hours, for example 10 to 24 hours, desirably at an elevated temperature such as 37 degrees C. Optionally, a mixing method such as sonication can be used during the soak. Additional processing steps can include decalcification, washing with water, and immersion in organic solvent solutions such as chloroform/methanol to remove cellular debris and sterilize. After such immersion the graft can be rinsed thoroughly with water and then frozen and optionally lyophilized. These and other conventional tissue preservation techniques can be applied to the osteochondral grafts or plugs in accordance with the present invention.

While certain discussions above have focused upon the use of harvested osteochondral grafts, in other aspects of the invention, grafts of and for use in the invention can be manufactured from other materials or components. Illustratively, grafts adapted for receipt in surgical openings in subchondral bone at articular sites, and desirably for integration with the subchondral bone, can be synthesized from natural or synthetic materials.

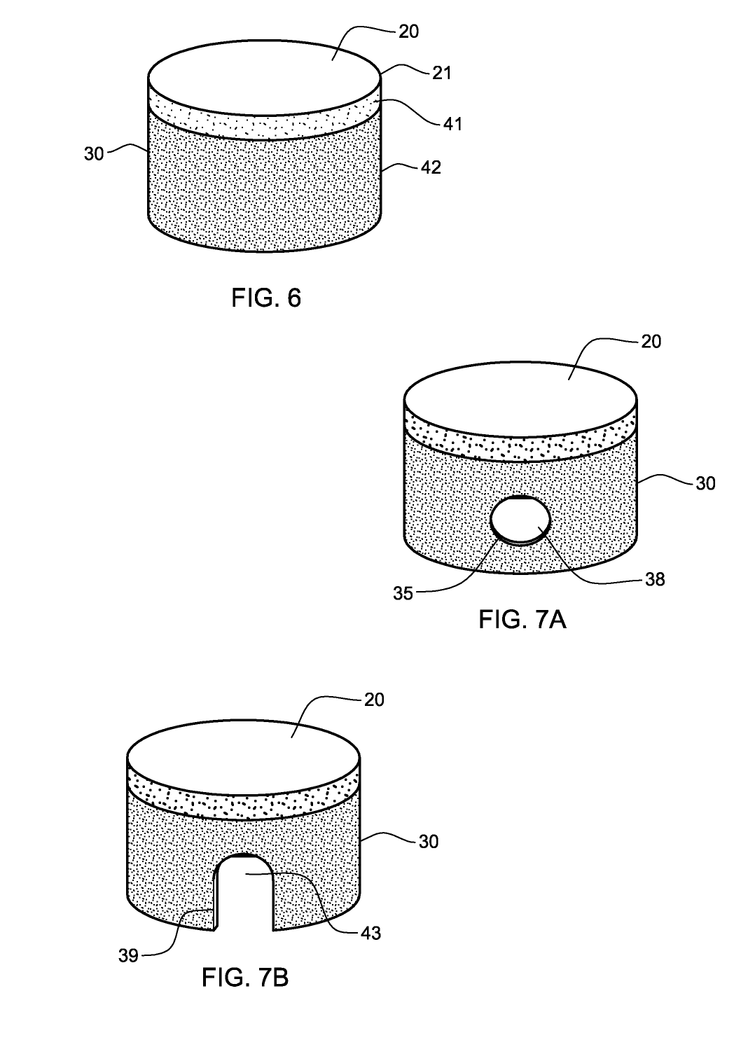

For example, as shown in FIG. 6, osteochondral graft 10 may be comprised of two materials providing a composite or biphasic construct. As depicted in FIG. 6, osteochondral graft 10 maybe fabricated from a collagen based material. The first collagen material 41 is more porous and less dense relative to the second collagen material 42 of the shown osteochondral graft. Cartilage cap 20 is shown as being fabricated from first collagen material 41 that has a level of porosity that results in cartilage cap 20 acting as a scaffold for new articular cartilage formation following implantation. Wall extension 30 is shown to be fabricated from second collagen material 42. Second collagen material 42 is substantially cross-linked and has a higher density in comparison to first material 41. The physical characteristics of second material 42 provides wall extension 30 with the necessary strength to withstand the forces applied to osteochondral graft 10 during the implantation procedure and post-operatively.

Further, it is contemplated that osteochondral grafts 10, 100, 200 can be synthesized from natural materials or from synthetic materials including bioabsorbable or non-bioabsorbable versions of these two types of materials. The natural materials may include, but are not limited to, collagen, chitosan, alginate, hyaluronic acid, silk, elastin, bone allograft, and osteochondral allograft, ceramics, or combinations thereof. Illustrative synthetic biocompatible materials, which may act as suitable matrices for portions of the osteochondral graft can include poly-alpha-hydroxy acids (e.g. polylactides, polycaprolactones, polyglycolides and their copolymers, such as lactic acid/glycolic acid copolymers and lactic acid/caprolactone copolymers), polyanhydrides, polyorthoesters, polydioxanone, segmented block copolymers of polyethylene glycol and polybutylene terephtalate (Polyactive), poly (trimethylenecarbonate) copolymers, tyrosine derivative polymers, such as tyrosine-derived polycarbonates, or poly (ester-amides). Suitable ceramic materials include, for example, calcium sulfate, calcium phosphate ceramics such as tricalcium phosphate, hydroxyapatite, and biphasic calcium phosphate. As discussed previously, the osteochondral grafts 10, 100, 200 may have a uniform composition throughout, or may vary, for instance having wall extension formed of a first, relatively strong and loadbearing material (e.g. a ceramic, polymer or composite), and cartilage cap formed of another material with the articulating surface formed by yet another material, for example a relatively smooth polymer layer. These and other variants would be apparent to the skilled artisan from the descriptions herein.

Various portions of the osteochondral graft invention can be used in conjunction with other materials helpful to the treatment. For example, the osteochondral grafts can be used in combination with a growth factor, and especially a growth factor that is effective in inducing formation of bone and/or cartilage tissue. Although not shown, such growth factors can be physically applied directly to bottom surface 22, internal surface 31, external surface 32, end 36, tab 34, hole 35, slot 39, or other parts, including concavities or reservoirs disposed on these listed structural elements of osteochondral graft 10, 100, 200 through dripping, spraying, dipping, or using a brush or other suitable applicator, such as a syringe. Alternatively, or in addition, amounts of the growth factor or other material(s) can be directly applied to opening 50, including platform 51 and channel 52, for example by filling or coating surgically-prepared opening 50 with one or more of these substances.

Desirably, the growth factor will be from a class of proteins known generally as bone morphogenic proteins (BMPs), and can in certain embodiments be recombinant human (rh) BMPs. These BMP proteins, which are known to have osteogenic, chondrogenic and other growth and differentiation activities, include rhBMP-2, rhBMP-3, rhBMP4 (also referred to as rhBMP-2B), rhBMP-5, rhBMP-6, rhBMP-7 (rhOP-1), rhBMP-8, rhBMP-9, rhBMP-12, rhBMP-13, rhBMP-15, rhBMP-16, rhBMP-17, rhBMP-18, rhGDF-1, rhGDF-3, rhGDF-5, rhGDF-6, rhGDF-7, rhGDF-8, rhGDF-9, rhGDF-10, rhGDF-11, rhGDF-12, rhGDF-14, TGFs, CDMPs, PDGF, PTM, and statins. For example, BMP-2, BMP-3, BMP-4, BMP-5, BMP-6 and BMP-7, disclosed in U.S. Pat. Nos. 5,108,922; 5,013,649; 5,116,738; 5,106,748; 5,187,076; and 5,141,905; BMP-8, disclosed in PCT publication WO91/18098; and BMP-9, disclosed in PCT publication WO93/00432, BMP-10, disclosed in U.S. Pat. No. 5,637,480; BMP-11, disclosed in U.S. Pat. No. 5,639,638, or BMP-12 or BMP-13, disclosed in U.S. Pat. No. 5,658,882, BMP-15, disclosed U.S. Pat. No. 5,635,372 and BMP-16, disclosed in U.S. Pat. Nos. 5,965,403 and 6,331,612. Other compositions which may also be useful include Vgr-2, and any of the growth and differentiation factors [GDFs], including those described in PCT applications WO94/15965; WO94/15949; WO95/01801; WO95/01802; WO94/21681; WO94/15966; WO95/10539; WO96/01845; WO96/02559 and others. Also useful in the present invention may be BIP, disclosed in WO94/01557; HP00269, disclosed in JP Publication number: 7-250688; and MP52, disclosed in PCT application WO93/16099. The disclosures of all of these patents and applications are hereby incorporated herein by reference. Also useful in the present invention are heterodimers of the above and modified proteins or partial deletion products thereof. These proteins can be used individually or in mixtures of two or more.

The BMP may be recombinantly produced, or purified from a protein composition. The BMP may be homodimeric, or may be heterodimeric with other BMPs (e.g., a heterodimer composed of one monomer each of BMP-2 and BMP-6) or with other members of the TGF-beta superfamily, such as activins, inhibins and TGF-beta 1 (e.g., a heterodimer composed of one monomer each of a BMP and a related member of the TGF-beta superfamily). Examples of such heterodimeric proteins are described for example in Published PCT Patent Application WO 93/09229, the specification of which is hereby incorporated herein by reference. The amount of osteogenic protein useful herein is that amount effective to stimulate increased osteogenic activity of infiltrating progenitor cells, and will depend upon several factors including the size and nature of the defect being treated, and the carrier and particular protein being employed. In certain embodiments, the amount of osteogenic protein to be delivered will generally be in a range of from about 0.05 to about 4.0 mg.

Additionally, the osteochondral graft of the invention may be coated in some fashion with an osteogenic protein used to form bone that can also be administered together with an effective amount of a protein which is able to induce the formation of tendon- or ligament-like tissue in the implant environment. Such proteins include BMP-12, BMP-13, and other members of the BMP-12 subfamily, as well as MP52. These proteins and their use for regeneration of tendon and ligament-like tissue are disclosed for example in U.S. Pat. Nos. 5,658,882, 6,187,742, 6,284,872 and 6,719,968, the disclosures of which are hereby incorporated herein by reference.

Further, the growth factor may be applied to the tissue source in the form of a buffered aqueous solution. Other materials which may be suitable for use in application of the growth factors in the methods and products of the present osteochondral graft invention include carrier materials such as collagen, milled cartilage, hyaluronic acid, polyglyconate, degradable synthetic polymers, demineralized bone, minerals and ceramics, such as calcium phosphates, etc., as well as combinations of these and potentially other materials.

Other biologically active materials may also be used in conjunction with osteochondral grafts of the present invention. These include for example cells such as human allogenic or autologous chondrocytes, human allogenic cells, human allogenic or autologous bone marrow cells, human allogenic or autologous stem cells and human allogenic or autologous osteocytes or osteoblasts, demineralized bone matrix, insulin, insulin-like growth factor-1, interleukin-1 receptor antagonist, hepatocyte growth factor, platelet-derived growth factor, and Indian hedgehog and parathyroid hormone-related peptide, to name a few. Again, such materials may be applied to osteochondral graft 10 or more specific portions of same, including bottom surface 22, internal surface 31, external surface 32, end 36, tab 34, hole 35 or slot 39. It is also contemplated that these example types of cells may also be applied to opening 50 and its related internal structures.

In certain modes of practice, suitable organic glue material may be used to help secure the osteochondral graft in place in the surgically created opening. Suitable organic glue material can be obtained commercially, such as for example; TISSEEL® or TISSUCOL® (fibrin based adhesive; Immuno AG, Austria), Adhesive Protein (Sigma Chemical, USA), Dow Corning Medical Adhesive B (Dow Corning, USA), fibrinogen thrombin, elastin, collagen, casein, albumin, keratin and the like.

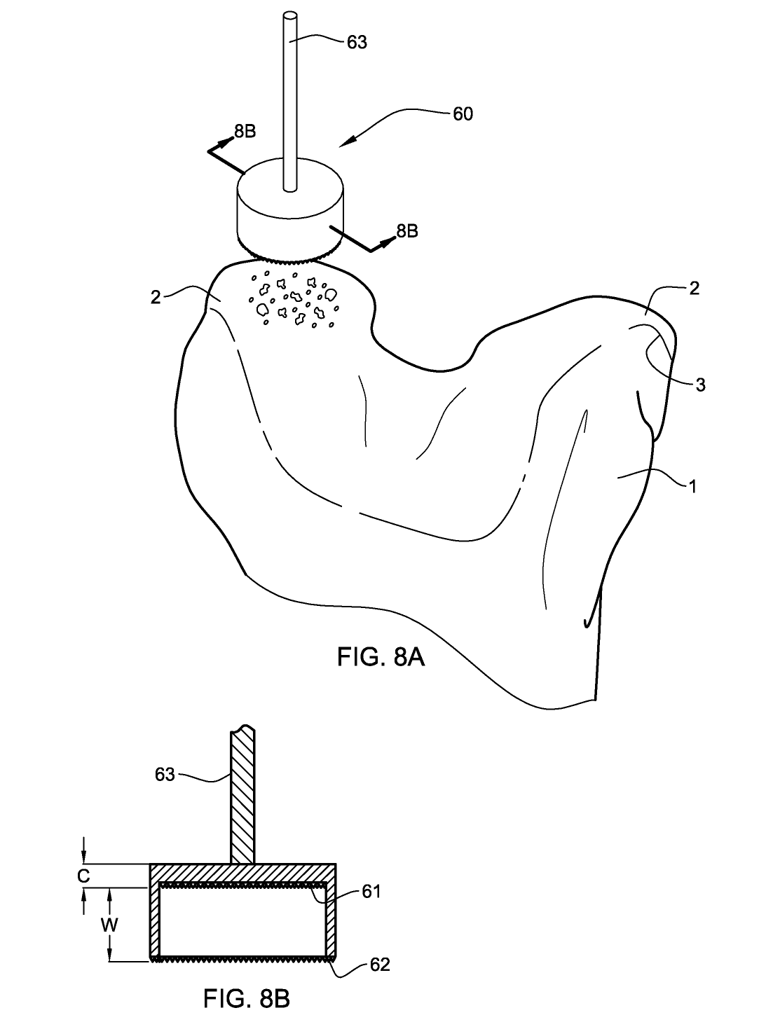

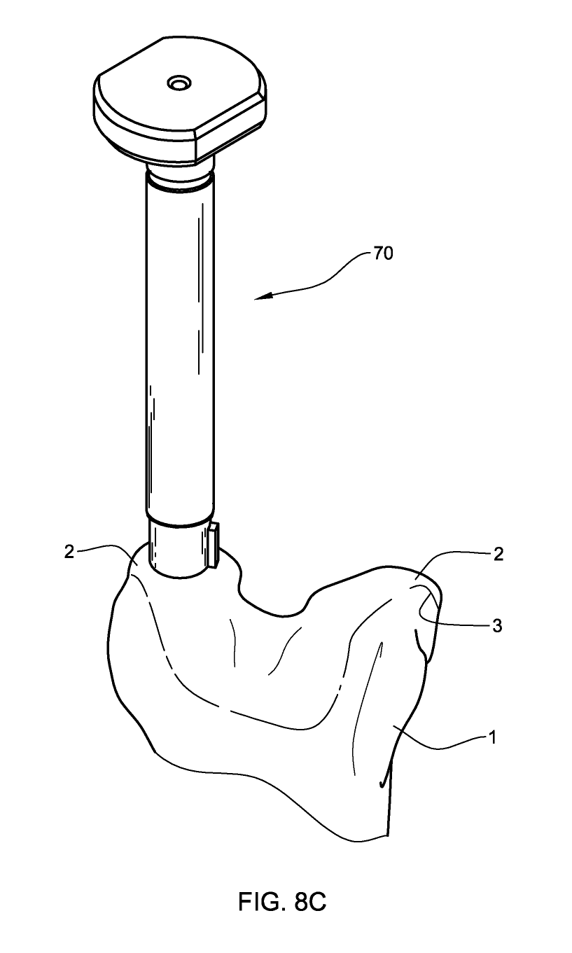

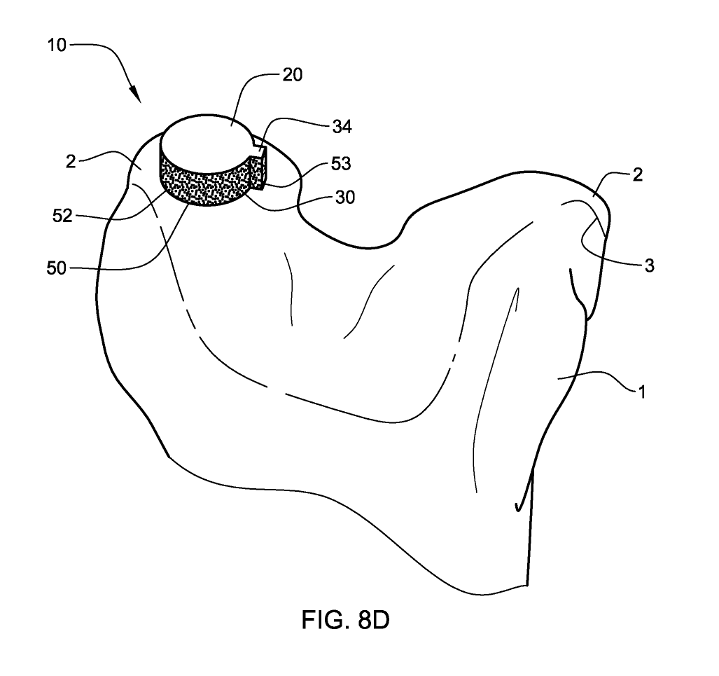

The method for repairing an articular cartilage defect site using an osteochondral graft is shown at FIGS. 8A-D. Initially, the user may surgically create an opening in the articular cartilage defect site by excising damaged or diseased cartilage and/or subchondral bone tissue at the site to create a hole or void in which osteochondral graft 10 will be received. Tissue removal can be conducted in any suitable manner including for instance drilling and/or punching, typically in a direction substantially perpendicular to the articular cartilage layer at the site, to create a void having a depth approximating that of the graft or plug to be implanted. Final preparation of the opening may be completed by using a coring tool 60 similar to the example shown in FIG. 8A.

FIG. 8B is a cross-sectional view of coring tool 60 showing the planar cutting surface 61 that is sized to remove the damaged articular cartilage. Planar cutting surface 61 includes a plurality of cutting teeth that when rotated by shank 63, remove cartilage and subchondral bone to create platform 51 that is positioned below or proximal to the joint surface. In addition, coring tool 60 has a series of peripheral cutting teeth 62 that extend a distance W from planar cutting surface 61. Distance W will generally correspond to the depth 56 of channel 152 that is equal to the length of wall extension 30. Peripheral cutting teeth 61 are configured to remove subchondral bone when rotated by shaft 63 resulting in the creation of channel 52 that is oriented and configured to receive wall extension 30. For other certain embodiments of the osteochondral graft invention, the extending side portions of peripheral cutting teeth 61 of coring tool 60 may include angular cutting members resulting in the tapered angled configurations of external surface 31 and internal surface 32 respectively, as has previously described herein.

As shown in FIG. 8C, surgically creating opening 50 includes further forming slot 53 along at least a portion of channel wall 54. This may be accomplished by using a punch tool 70 that is generally centralized within opening 50 by mating with previously formed platform 51.

The method also includes employing or obtaining osteochondral graft 10 that includes cartilage cap 20 and wall extension 30. Wall extension 30 will generally have tab 34 disposed along external surface 31 for post-operative rotational control. The structural features of osteochondral graft 10 have been discussed previously herein and for brevity sake, will not be repeated here.

Generally, the method provides further for the implantation of osteochondral graft 10 into the prepared opening 50 as seen in FIG. 8D. Final seating of osteochondral graft within opening 50 results in top surface 21 of cartilage cap 20 being positioned tangent or flush to the adjacent remaining articular cartilage 3 of the patient. Bottom surface 22 will also be positioned to contact platform 51 to provide structural support to cartilage cap 20 to eliminate post-operative collapse of top surface 21 and to facilitate bio-ingrowth of osteochondral graft 10 to the host defect site. Also during implantation, wall extension 30 is slid into channel 52 with tab 34 being aligned with slot 53 to ensure rotational stability of the graft or plug post-operatively.

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the following claims.

What is claimed is:

1. An osteochondral graft for repairing an articular cartilage defect site, the osteochondral graft comprising:
a cartilage cap and a wall extension, wherein the cartilage cap has a top surface and a bottom surface, and the wall extension projects from the bottom surface of the cartilage cap;
wherein the wall extension comprises an external surface and an internal surface, with the internal surface at least partially bounding an internal space; and
wherein the wall extension and internal space are configured to facilitate implantation of the osteochondral graft into the articular cartilage defect site.

2. The osteochondral graft of claim 1, wherein the osteochondral graft further comprises a central axis extending between the cartilage cap and an end of the wall extension.

3. The osteochondral graft of claim 2, wherein one of the internal surface or external surface is tapered at a taper angle relative to the central axis, the taper angle facilitating insertion of the osteochondral graft into the articular cartilage defect site.

4. The osteochondral graft of claim 1, wherein the osteochondral graft is fabricated from a natural material, the natural material facilitating bio-ingrowth of the osteochondral graft within the articular cartilage defect site.

5. The osteochondral graft of claim 4, wherein the natural material is at least one of chitosan, alginate, hyaluronic acid, silk, elastin, bone allograft and osteochondral allograft.

6. The osteochondral graft of claim 4, wherein the natural material is collagen.

7. The osteochondral graft of claim 4, wherein the natural material is one of at least bioabsorbable or non-bioabsorbable.

8. The osteochondral graft of claim 6, wherein the osteochondral graft is configured as a composite collagen construct comprising a first material and a second material, and the cartilage cap comprises the first material, the first material being a porous collagen, the porous collagen being a scaffold for new cartilage formation, and wherein the wall extension comprises the second material, the second material being a substantially cross-linked collagen, the substantially cross-linked collagen facilitating insertion of the wall extension into the articular cartilage defect site.

9. The osteochondral graft of claim 6, wherein the first material and the second material each have a density, wherein the density of the second material is greater than the density of the first material.

10. The osteochondral graft of claim 1, wherein the osteochondral graft is fabricated from a biocompatible synthetic material, the biocompatible synthetic material facilitating bio-ingrowth of the osteochondral graft within the articular cartilage defect site.

11. The osteochondral graft of claim 10, wherein the biocompatible synthetic material is at least one of calcium phosphate ceramic, calcium sulfate, poly-alpha-hydroxyl acid, polyanhydrides, polyorthoesters, polydioxanone, segmented block copolymers of polyethylene glycol, polybutylene terephtalate copolymers and tyrosine derivative polymers.

12. The osteochondral graft of claim 10, wherein the biocompatible synthetic material is one of at least bioabsorbable or non-bioabsorbable.

13. The osteochondral graft of claim 1, wherein the wall extension further comprises at least one radially projecting tab disposed on the external surface, wherein the at least one radially projecting tab extends at least partially along the length of the external surface and is configured to fit into a corresponding at least one slot disposed within the articular cartilage defect site to mechanically interlock the osteochondral graft with the articular cartilage defect site to prevent rotational movement of the osteochondral graft after implantation.

14. The osteochondral graft of claim 1, wherein the wall extension further comprises at least one opening, the at least one opening extending from the internal surface to the external surface and being sized to facilitate bio-ingrowth into the wall extension of the osteochondral graft.

15. The osteochondral graft of claim 14, wherein the at least one opening is at least one of a hole and a slot.

16. The osteochondral graft of claim 1, wherein a growth factor is disposed on the osteochondral graft, the growth factor facilitating bio-ingrowth of the osteochondral graft into the articular cartilage defect site.

17. The osteochondral graft of claim 16, wherein the growth factor is selected from the group consisting of BMP-2, BMP-7, GDF-5, TGF, PDGF and statin.

18. The osteochondral graft of claim 1, wherein the wall extension has a thickness between the internal surface and external surface, and the wall extension thickness has a value ranging from 1 to 5 millimeters, and wherein the cartilage cap has a thickness between the top surface and bottom surface, and the cartilage cap thickness has a value ranging from 2 to 5 millimeters.

19. The osteochondral graft of claim 3, wherein taper angle has a value ranging from 2 to 10 degrees.

20. The osteochondral graft of claim 1, wherein the osteochondral graft has a cross-sectional shape, the cross-sectional shape being one of a circle, an oval, or a polygonal shape, wherein the cross-sectional shape of the osteochondral graft is configured to fit within the articular cartilage defect site.

21. The osteochondral graft of claim 1, wherein the articular cartilage defect site comprises a centrally positioned platform and a channel, the channel being at least partially circumferential relative to the platform and extending to a depth below the articular cartilage defect site, and wherein the articular cartilage defect site is configured to receive the osteochondral graft with the bottom surface of the cartilage cap contacting the platform of the articular cartilage defect site and the wall extension sliding into the channel of the articular cartilage defect site, thereby facilitating bio-ingrowth between the osteochondral graft and the articular cartilage defect site.

22. An osteochondral graft for repairing an articular cartilage defect site, the osteochondral graft comprising:
a cartilage cap and a wall extension, wherein the cartilage cap has a top surface and a bottom surface, and the wall extension projects from the bottom surface of the cartilage cap;
wherein the wall extension comprises an external surface and an internal surface, with the internal surface at least partially bounding an internal space;
wherein the osteochondral graft further comprises a central axis extending between the cartilage cap and an end of the wall extension; and
wherein one of the internal surface or external surface is tapered at a taper angle relative to the central axis, the taper angle facilitating insertion of the osteochondral graft into the articular cartilage defect site.

23. The osteochondral graft of claim 22, wherein the osteochondral graft is fabricated from a natural material, the natural material facilitating bio-ingrowth of the osteochondral graft within the articular cartilage defect site.

24. The osteochondral graft of claim 23, wherein the natural material is at least one of chitosan, alginate, hyaluronic acid, silk, elastin, bone allograft and osteochondral allograft.

25. The osteochondral graft of claim 23, wherein the natural material is collagen.

26. The osteochondral graft of claim 23, wherein the natural material is one of at least bioabsorbable or non-bioabsorbable.

27. The osteochondral graft of claim 22, wherein the osteochondral graft is configured as a composite collagen construct comprising a first material and a second material, and the cartilage cap comprises the first material, the first material being a porous collagen with a first density, the porous collagen being a scaffold for new cartilage formation, and wherein the wall extension comprises the second material, the second material being a substantially cross-linked collagen with a second density, the substantially cross-linked collagen facilitating insertion of the wall extension into the articular cartilage defect site, and wherein the second density of the second material is greater than the first density of the first material.

28. The osteochondral graft of claim 22, wherein the osteochondral graft is fabricated from a biocompatible synthetic material, the biocompatible synthetic material facilitating bio-ingrowth of the osteochondral graft within the articular cartilage defect site.

29. The osteochondral graft of claim 28, wherein the biocompatible synthetic material is at least one of calcium phosphate ceramic, calcium sulfate, poly-alpha-hydroxyl acid, polyanhydrides, polyorthoesters, polydioxanone, segmented block copolymers of polyethylene glycol, polybutylene terephtalate copolymers and tyrosine derivative polymers.

30. The osteochondral graft of claim 28, wherein the biocompatible synthetic material is one of at least bioabsorbable or non-bioabsorbable.

31. The osteochondral graft of claim 22, wherein the wall extension further comprises at least one radially projecting tab disposed on the external surface, wherein the at least one radially projecting tab extends at least partially along the length of the external surface and is configured to fit into a corresponding at least one slot disposed within the articular cartilage defect site to mechanically interlock the osteochondral graft with the articular cartilage defect site to prevent rotational movement of the osteochondral plug after implantation.

32. The osteochondral graft of claim 22, wherein the wall extension further comprises at least one opening, the at least one opening extending from the internal surface to the external surface and being sized to facilitate bio-ingrowth into the wall extension of the osteochondral graft.

33. The osteochondral graft of claim 32, wherein the at least one opening is at least one of a hole and a slot, and a slit.

34. The osteochondral graft of claim 22, wherein a growth factor is disposed on the osteochondral graft, the growth factor facilitating bio-ingrowth of the osteochondral graft into the articular cartilage defect site.

35. The osteochondral graft of claim 34, wherein the growth factor is selected from the group consisting of BMP-2, BMP-7, GDF-5, TGF, PDGF and statin.

36. The osteochondral graft of claim 22, wherein the wall extension has a thickness between the internal surface and external surface, and the wall extension thickness has a value ranging from 1 to 5 millimeters, and wherein the cartilage cap has a thickness between the top surface and bottom surface, and the cartilage cap thickness has a value ranging from 2 to 5 millimeters.

37. The osteochondral graft of claim 22, wherein the taper angle has a value ranging from 2 to 10 degrees.

38. The osteochondral graft of claim 22, wherein the articular cartilage defect site comprises a centrally positioned platform and a channel, the channel being at least partially circumferential relative to the platform and extending to a depth below the articular cartilage defect site, and wherein the articular cartilage defect site is configured to receive the osteochondral graft with the bottom surface of the cartilage cap contacting the platform of the articular cartilage defect site and the wall extension sliding into the channel of the articular cartilage defect site, thereby facilitating bio-ingrowth between the osteochondral graft and the articular cartilage defect site.

39. A method of repairing an articular cartilage defect site in a mammal, the method comprising:
surgically creating an opening in the articular cartilage defect site, wherein the opening comprises a centrally positioned platform and a channel, the channel being at least partially circumferential relative to the platform and extending to a depth below the articular cartilage defect site;
employing an osteochondral graft, the osteochondral graft comprising:
a cartilage cap and a wall extension, wherein the cartilage cap has a top surface and a bottom surface, the wall extension projecting from the bottom surface of the cartilage cap and comprising an external surface and an internal surface, wherein the internal surface at least partially bounds an internal space; and
implanting the osteochondral graft into the opening, wherein the osteochondral graft is sized and configured to be inserted into the surgically created opening, wherein when inserted, the bottom surface of the cartilage cap contacts the platform of the opening and the wall extension slides into the channel of the opening to facilitate bio-ingrowth between the osteochondral graft and the opening.

40. The method of claim 39, wherein the surgically creating an opening further comprises employing a coring tool for removing the articular cartilage defect and a portion of the subchondral bone, the coring tool comprising a planar cutting surface integrally attached to peripheral cutting teeth, the planar cutting surface being sized to correspond to the articular cartilage defect site, wherein the peripheral cutting teeth extend a distance from the planar cutting surface corresponding to a length of the osteochondral graft and a depth of the surgically created opening.

41. The method of claim 40, wherein the planar cutting surface of the coring tool is sized and configured to remove a portion of the articular cartilage from the articular cartilage defect site, thereby creating the opening and the platform, and wherein the peripheral cutting teeth of the coring tool are sized and configured to remove a portion of subchondral bone positioned within the articular cartilage defect site, thereby creating the channel circumferential relative to the platform and sized to receive the wall extension of the osteochondral graft after implantation of the osteochondral graft.

42. The method of claim 39, wherein the surgically creating an opening further comprises employing a punch tool for forming at least one slot, the at least one slot being disposed within at least one side wall of the channel of the opening.

43. The method of claim 42, wherein the wall extension of the osteochondral graft further comprises at least one radially projecting tab disposed on the external surface, and wherein the at least one radially projecting tab extends at least partially along the length of the external surface and is configured to fit into the corresponding at least one slot disposed within at least one side wall of the channel to mechanically interlock the osteochondral graft within the opening to prevent rotational movement of the osteochondral graft after implantation of the osteochondral graft.

44. The method of claim 39, wherein the channel of the opening is concentric relative to the centrally positioned platform of the opening to facilitate the implanting of the osteochondral graft into the opening.

45. The method of claim 39, wherein the implanting further comprises implanting a first osteochondral graft into a first opening and implanting a second osteochondral graft into adjacent second opening, wherein the first and second osteochondral grafts, as implanted are interlocked to substantially inhibit movement of the first osteochondral graft and the second osteochondral graft after implantation in the articular cartilage defect site.
